(19) United States
(12) Patent Application Publication
(10) Pub. No.: US 2005/0205108 A1
(43) Pub. Date: Sep. 22, 2005

(54) Method and system for immersion lithography lens cleaning

(75) Inventors: Ching-Yu Chang, Yen-Sun (TW); Chin-Hsiang Lin, Hsin-Chu (TW)

Correspondence Address: DUANE MORRIS, LLP;IP DEPARTMENT, ONE LIBERTY PLACE, PHILADELPHIA, PA, 19103-7396, US

(73) Assignee: Taiwan Semiconductor Manufacturing Co., Ltd.

(21) Appl. No.: 10/802,087

(22) Filed: Mar. 16, 2004

(51) Int. Cl. B08B 3/12; G21G 5/00; G03B 27/42; B08B 7/02; A61N 5/00; B08B 6/00; B08B 7/00

(52) U.S. Cl. 134/1; 355/53

(57) ABSTRACT

A method and system for cleaning lens used in an immersion lithography system is disclosed. After positioning a wafer in the immersion lithography system, a light exposing operation is performed on the wafer using an objective lens immersed in a first fluid containing surfactant, wherein the surfactant reduces a likelihood for having floating defects adhere to the wafer and the objective lens.

### BACKGROUND

The present disclosure relates generally to immersion lithography processes used for the manufacture of semiconductor devices, and more particularly, to the cleaning of the lens used within the immersion lithography systems.

The manufacture of very large-scale integrated (VLSI) circuits require the use of many photolithography process steps to define and create specific circuits and components onto the semiconductor wafer (substrate) surface. Conventional photolithography systems comprise several basic subsystems, a light source, optical transmission elements, transparent photo mask reticles, and electronic controllers. These systems are used to project a specific circuit image, defined by the mask reticle, onto a semiconductor wafer coated with a light sensitive film (photoresist) coating. As VLSI technology advances to higher performance, circuits become geometrically smaller and denser, requiring lithography equipment with lower resolution projection and printing capability. Such equipment is required to be capable of resolutions lower than 100 nanometers (nm). As new device generations are developed requiring even further improvements, with feature resolutions 65 nm and lower, major advancements to photolithography processing were required.

Immersion lithography has been implemented to take advantage of the process technology's capability for much improved resolution. Immersion lens lithography features the usage of a liquid medium to fill the entire gap between the last objective lens of the light projection system and the semiconductor wafer (substrate) surface during the light exposure operations of the photoresist pattern printing process. The liquid immersion medium of the immersion lens lithographic technique provides an improved index of refraction for the exposing light, thus improving the resolution capability of the lithographic system. This is shown with the Rayleigh Resolution formula, R=kλ/N.A., where R (resolution) is dependant upon k (certain process constants), λ (wavelength of the transmitted light), and the N.A. (Numerical Aperture of the light projection system). It is noted that N.A. is a function of the index of refraction, N.A.=n sinθ, where n is the index of refraction of the liquid medium between the objective lens and the wafer substrate, and θ is the acceptance angle of the lens for the transmitted light.

It can be seen that as the index of refraction (n) becomes higher for a fixed acceptance angle, the numerical aperture (N.A.) of the projection system becomes larger, thus providing a lower resolution (R) capability for the lithographic system. Conventional immersion lithographic systems utilize de-ionized water as the immersion lens fluid between the objective lens and the wafer substrate. De-ionized water at 20 degrees Celsius has an index refraction of approximately 1.33 versus air which has an index refraction of approximately 1.00. It can be seen that immersion lithographic systems utilizing de-ionized water as the immersion lens fluid, offers much improvement to the resolution capability of the photolithography processes.

FIG. 1 is a cross-sectional diagram that illustrates the typical immersion lithography process. The immersion printing section 100 of the lithography system shows a wafer stage 102 with a photoresist coated wafer 104 located on top of the wafer stage. The de-ionized water immersion lens 106 is shown located on top of the photoresist coated wafer 104, comprising of the water immersion lens fluid and displacing the entire volume of space between the wafer and the last objective lens 108 of the lithography's light projection system 118. The fluid of the water immersion lens 106 is in direct contact with both the top surface of the photoresist coated wafer 104 and the lower surface of the objective lens 108. It is noted that if a photoresist protective layer (not shown) is used, it would be located between the top surface of the photoresist coated wafer 104 and in direct contact to the water immersion lens 106.

There are two fluid reservoirs directly connected to the fluid of the water immersion lens 106. The fluid supply reservoir 112 serves as the means for supplying and dispensing the water immersion lens fluid to the water immersion lens 106. The fluid recovery reservoir 114 serves as the means for recovering and accepting the output fluid flow from the water immersion lens 106. It is noted that the water immersion fluid flow is in the direction starting from the fluid supply reservoir 112, through the water immersion lens 106, and out to the fluid recovery reservoir 114. There may be associated mechanical hardware and electrical/electronic controllers by which the flow of water immersion lens fluid as described above, is managed and controlled. The large downward arrow 110 of FIG. 1 located above the lithography system's last objective lens 108 represents the direction and transmission of the pattern image-exposing light towards the objective lens and through the water immersion lens 106.

The use of de-ionized water as the immersion fluid in typical immersion lithography systems imposes certain concerns for the process operations. The photoresist layer on top of the wafer substrate 104 may have certain tendencies to outgas, producing gas micro-bubbles within the water immersion lens 106 during the photolithographic printing; same being enough to distort the printed pattern and disturb the contrast of the printed images. The water of the immersion lens 106, enhanced by the applied photo energy upon the photoresist layer during the light exposing operations and the absorption of water by the photoresist, may also induce the dissociation and breakdown of the photoresist layer, to cause particulate contamination into the water immersion lens fluid. The micro-bubbles and photoresist particulates may then flow within the water immersion lens 106 to eventually settle and adhere onto the immersion fluid interfaces. Additional general particulate contamination, originally from sources such as the environment, hardware components and any already existing as incoming on the associated materials, may become free floating within the water immersion lens 106 to also eventually settle and adhere onto the immersion fluid interfaces.

The physical particulate defects, like the gas micro-bubbles, may subsequently distort the printed pattern and disturb the contrast of the printed images. The defects may further complicate the pattern printing processes via interference with the flow of the water immersion lens fluid. The high viscosity of the de-ionized water may cause stagnancy issues with the fluid flows and temperature uniformities within the immersion lens. As a result, stagnant fluid and localized heat spots may induce undesired physical and chemical effects to and within the immersion fluid, photoresist interface, as well as to the immersion fluid, and objective lens interface. The immersion fluid, and photoresist interfaces are particularly an issue as its hydrophobic (water repelling) surface property readily allows for the defects and gas micro-bubbles to migrate and adhere upon its surface.

FIG. 2 illustrates the gas micro-bubble and particulate defect concerns associated with the water immersion lens fluid and its material interfaces. FIG. 2 is a cross-sectional diagram 200 showing close views of the water immersion lens 106 and the fluid's interfaces to the contacted wafer's photoresist layer 104 and to the contacted objective lens 108 of the immersion lithography system. The fluid of the water immersion lens 106 is in direct contact to the lowest surface of the objective lens 108 at the interface labeled 208 on the diagram. The fluid of the water immersion lens 106 is in direct contact to the top surface of the photoresist (or photoresist protective) layer 104 at the interface labeled 210 on the diagram. The water immersion fluid flow is depicted by the two horizontal arrows drawn pointing towards the left. The right most arrow pointing left indicates the flow of water immersion lens fluid into the immersion lens 106. The left most arrow pointing left indicates the flow of fluid out of the water immersion lens 202. It shows three defect types located within the water immersion lens 202. There are resist defects (R), micro-bubbles (B) and general, miscellaneous particulates (P). Some of the described defects, R, B and P, are free, floating within the water immersion lens 202. Other defects, R, B and P are shown adhering to the two immersion lens interfaces, 208 and 210. It is noted that many of the adhered defects may have strong enough adhering forces that may not be overcome and released by the applied forces of the incoming flow of fluid. As a result, such defects may continue to grow and build, to become large enough to distort and disturb the quality of the printed pattern upon the photoresist.

To help prevent and minimize such defect mechanisms and issues from affecting the immersion lithography processes, certain semiconductor manufacturing facilities and technologies may implement at least one of several solutions. One solution requires an additional, thin transparent protective layer on top of the photoresist layer. Such protective layer serves as a mechanical barrier to minimize the photoresist contact with the water of the immersion lens fluid. The barrier suppresses and possibly stops the migration of water to the resist and thus the subsequent dissociation/breakdown of the resist and formation of gas micro-bubbles. Such method of protection is effective only to a certain extent and requires much additional production materials, production equipment, invested labor and time costs to implement within the manufacturing facilities and operations. Other solutions may be more hardware related to the immersion lithography system. Procedures requiring the halt of production to perform mechanical breakdown of components so that cleaning procedures may be performed; such that these procedures or preventive maintenance operations are costly and time consuming, requiring manpower, materials and the loss of equipment time for production usage.

Other facilities may implement the use of liquid filtration media within the water immersion fluid circulation and distribution loops. The filtration may decrease performance to the immersion lithography system as the water immersion fluid flow is decreased, reducing its heat transfer capabilities. Some facilities may implement optical filters within the immersion lithography system in an attempt to optically filter out and/or defocus the effects of the defects' imaging upon the photoresist. Such optical filters may also decrease performance of the immersion lithography system by compromising such qualities as the resolution, contrast and uniformities of the printed image patterns. These filtration methods have shown to add significant time and costs to the production operations, in addition to the requirement for sacrifice to some performance areas of the processes' product.

What is desired is an improved method for cleaning the lens used within the immersion lithography systems.

### SUMMARY

In view of the foregoing, this disclosure provides an improved method and system for cleaning lens used in an immersion lithography system. After positioning a wafer in the immersion lithography system, a light exposing operation is performed on the wafer using an objective lens immersed in a first fluid containing surfactant, wherein the surfactant reduces a likelihood for having floating defects adhere to the wafer and the objective lens.

The disclosed method and system minimizes the adhesion, formation and growth of particulate defects within the immersion lithography system.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 illustrates a cross-sectional view of the conventional immersion lithography process.

FIG. 2 illustrates a cross-sectional view of the conventional immersion lithography process with the locations of the various fluid, material interfaces and the defect types generated during the immersion lithography processes.

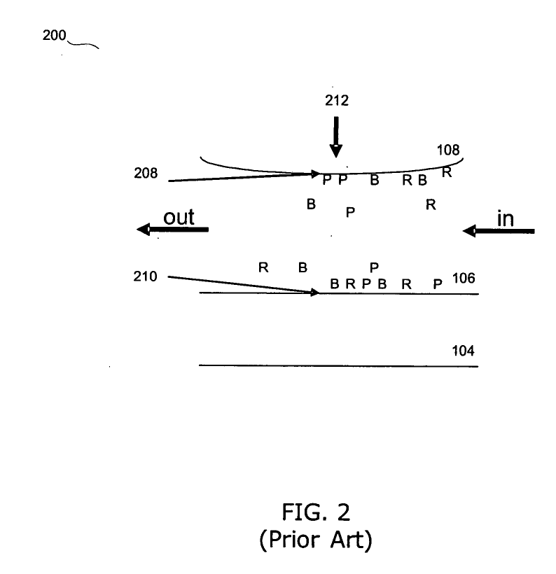

FIG. 3 illustrates a cross-sectional view of the disclosed immersion lithography cleaning process with the locations of the various fluid-material interfaces and the defect types within a surfactant-spiked imiersion lens fluid, during the immersion lithography processes.

FIG. 4 illustrates a cross-sectional view of the disclosed immersion lithography process configured for the disclosed cleaning method utilizing a separate surfactant-spiked cleaning solution system.

FIGS. 5a and 5b are two flow charts to summarize the sequence of procedural steps for the implementation of the two examples of the disclosed lens cleaning methods utilizing surfactant-spiked solutions.

### DESCRIPTION

The present disclosure describes an improved method for the cleaning of the lens used within the immersion lithography systems. The disclosed method uses surfactant-spiked solutions to maintain clean water immersion lens fluids in addition to clean fluid-material interfaces. The present disclosure provides several examples of applying the disclosed surfactant-spiked solutions The adhesion, formation and growth of particulate defects upon the immersion fluid, photoresist interface, as well as to the immersion fluid, objective lens interface are cleaned and/or kept clean.

The water immersion lens fluid of the present disclosure uses fluids with low, spiked concentrations of an added surfactant. Such surfactant may be either ionic (anionic or cationic) or non-ionic in nature. The surfactant modifies the properties of the water immersion lens fluid such that the surface and interfacial tension forces within the water immersion lens are greatly reduced. As a result, the surfactant-spiked fluid acts as a wetting agent (or detergent) to maintain the defect particulates and gas micro-bubbles suspended in the water immersion fluid as well as maintaining the immersion lens, photoresist interface and the immersion lens, objective lens interface free of any adhering particulates and gas micro-bubbles. The free floating particulates and micro-bubbles are subsequently purged away from the water immersion lens through the flow of water immersion lens fluid from the supply reservoir, through to the recovery reservoir.

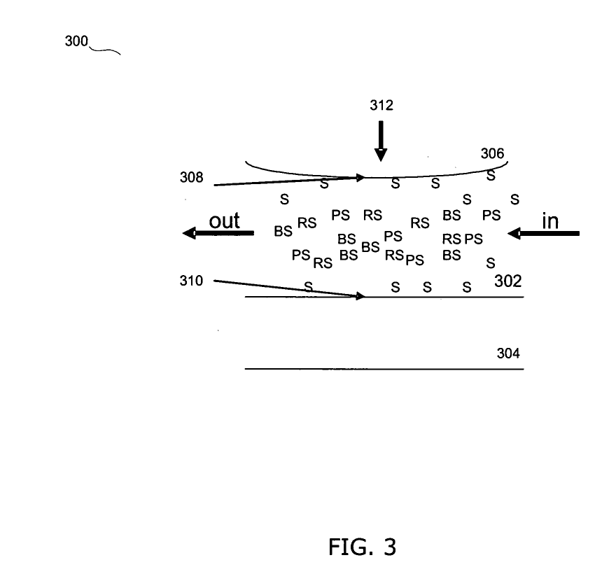

FIG. 3 is a cross-sectional diagram to illustrate the effects of the surfactant-spiked water immersion lens fluid during the immersion lithography processing. It shows close views of the water immersion lens 302, the fluid's interface to the contacted wafer's photoresist layer 304, and to the contacted objective lens 306 of the immersion lithography system. The fluid of the water immersion lens 302 is in direct contact to the lowest surface of the objective lens 306 at the interface labeled 308 on the diagram. The fluid of the water immersion lens 302 is in direct contact to the top surface of the photoresist (or photoresist protective) layer 304 at the interface labeled 310 on the diagram. The water immersion fluid flow is depicted by the two horizontal arrows drawn pointing towards the left. The right most arrow pointing left indicates the flow of water immersion lens fluid into the immersion lens 302. The left most arrow pointing left indicates the flow of fluid out of the water immersion lens 302. It also shows the surfactant molecules (S) and the three defect types located within the water immersion lens 302. There are resist defects (R), micro-bubbles (B) and general, miscellaneous particulates (P). The surfactant molecules (S) are shown free floating within the water immersion lens 302, as well as located at the two immersion lens interfaces, 308 and 310. The surfactant, at these two interfaces 308 and 310, has modified the interfaces such that they are wetted, with water of low surface tension. As a result, there are not strong enough forces between any of the gas micro-bubbles or free floating particulates to adhere to these interfaces. It is also noted that the wetting of the photoresist (or photoresist protective layer) surface 304 modifies the surface property such that it becomes more hydrophilic (affinity for water) in nature, rather than hydrophobic.

The described defects, R, B and P, are free floating within the fluid of the water immersion lens 302. These defects, R, B and P are wetted by the surfactant S, maintaining the defects within the main body of the fluid instead of migrating and adhering to the two immersion lens interfaces, 308 and 310. It is noted that the wetted defects do not have strong enough forces to adhere to either the immersion fluid, photoresist interface 310, or to the immersion fluid, objective lens interface 308. The wetted defects are thus overcome by and eventually purged from the water immersion lens 306 by the forces of the incoming flow of immersion lens fluid. As a result, there is neither growth nor buildup of defects, to become large enough to distort and disturb the quality of the printed pattern upon the photoresist.

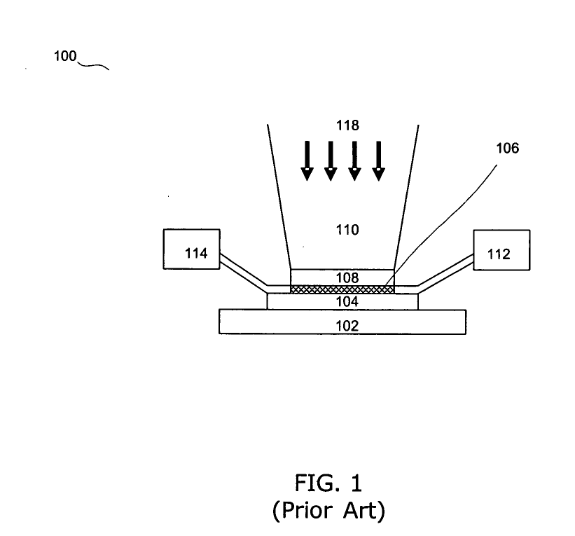

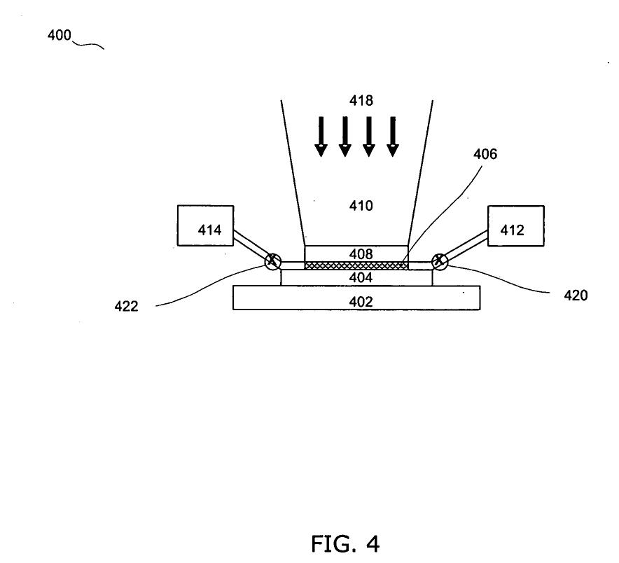

FIG. 4 illustrates a cross-sectional view of the disclosed immersion lithography process configured for another example of the disclosed cleaning method utilizing a surfactant-spiked cleaning solution. The disclosed immersion lithography printing section 400 is similar to the conventional system as previously described for FIG. 1. There is the wafer stage 402 with a photoresist coated wafer 404 located on top of the wafer stage. The de-ionized water immersion lens 406 is shown located on top of the photoresist coated wafer 404, comprised of the water immersion lens fluid displacing the entire volume of space between the wafer and the last objective lens 408 of the lithography's light projection system 418. There are the two fluid reservoirs directly connected to the fluid of the water immersion lens 406. The fluid supply reservoir 412 serves as the means for supplying and dispensing the water immersion lens fluid to the water immersion lens 406. The fluid recovery reservoir 414 serves as the means for recovering and accepting the output fluid flow from the water immersion lens 406. The water immersion fluid flow is shown with the direction starting from the fluid supply reservoir 412, through the water immersion lens 406, and out to the fluid recovery reservoir 414. The large downward arrow 410 of FIG. 4 located above the lithography system's last objective lens 408 represents the direction of, and the transmission of, the pattern image-exposing light towards the objective lens and through the water immersion lens 406.

The configuration of the improved immersion lithography printing section 400 features additional plumbing, related hardware and controller systems for additional fluid paths to the water immersion lens 406. FIG. 4 shows an input valve 420 located between the primary immersion lens fluid supply reservoir 412 and the water immersion lens 406. This input valve 420 and its associated controls and hardware, allows for optional fluid to be flowed from a secondary supply reservoir (not shown) into the immersion lens area 406. There is a corresponding output valve 422 with its related hardware and control system located between the output of the water immersion lens 406 and the primary immersion lens fluid recovery reservoir 414. This output valve 422 and its associated controls and hardware allows for the optional fluid dispensed by the disclosed input valve 420 to be recovered separately into a secondary recovery reservoir (not shown), outside of the primary recovery reservoir 414.

The disclosed valves, and control systems 420 and 422 for the secondary supply and recovery reservoirs allow for the combined use of water immersion lens fluid without surfactant, as well as the use of surfactant-spiked water immersion fluids. The input 420 and output 422 valves at the input and outputs of the water immersion lens 406 allow for the process users to implement and manipulate at least two different immersion lens fluids. As an example, the immersion lens lithography operations may be set up such that non-surfactant immersion lens fluid may be used during the actual immersion lithography printing/exposure operations. An optional surfactant-spiked immersion lens fluid may subsequently flow into the water immersion lens 406 and partially recovered via the secondary valved supply/recovery reservoirs. The optional surfactant-spiked immersion lens fluid may be used to perform the purging and cleaning of the water immersion lens 406 of the undesired gas micro-bubbles and particulate defects. Such cleaning technique may also include additional procedures, hardware and controls to provide additional rinses or purges with at least another fluid. Such rinses may include such fluids as ozonated water or hydrogen peroxide mixed with water.

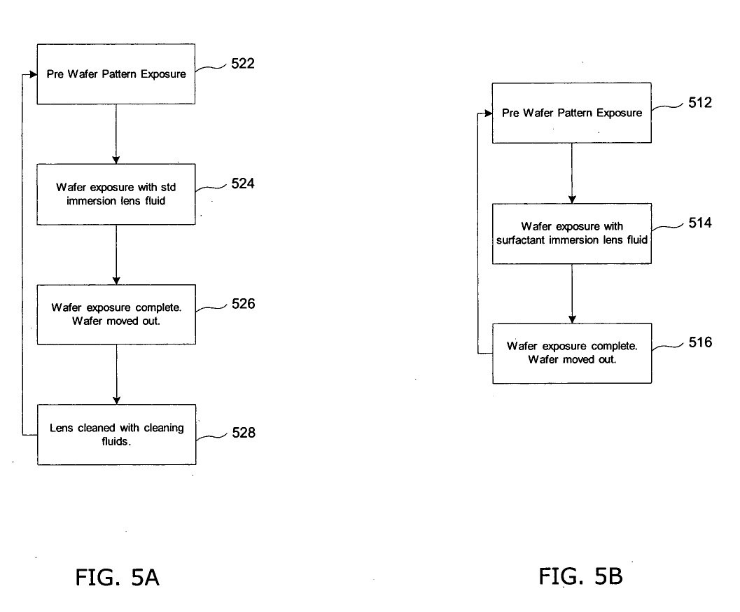

The flow diagrams of FIGS. 5a and 5b summarize the sequence of procedural steps for the implementation of the two described examples of the disclosed lens cleaning methods utilizing surfactant-spiked solutions. FIG. 5a summarizes the procedural steps for the disclosed example where surfactant-spiked immersion lens fluid is used as the water immersion lens during the actual light exposing printing operations. The preparation and setup of the photoresist coated wafer onto the immersion lithography system is performed as the first step 512 of the diagram. The light exposing operation to print the image pattern onto the photoresist of the production wafer is performed as the next step 514. The immersion lithography printing is complete and the wafer is moved out of the immersion lens lithography system as step 516 of the diagram. The immersion lithography system proceeds to begin processing the next wafer as the diagram points back to restart with step 512. The immersion lens lithography system of this example diagram, FIG. 5a, utilizes a surfactant-spiked water immersion lens fluid to replace the use of the standard non surfactant-spiked water immersion lens fluid of a conventionally configured system.

FIG. 5b summarizes the procedural steps for the disclosed example where surfactant-spiked water immersion lens fluid is used as the lens cleaner and where non surfactant-spiked water immersion lens fluid is used during the actual light exposing printing operations. It is noted that the disclosed system configuration featuring the added input and output valves and secondary reservoir systems as described for and by FIG. 4 is applicable for the procedures either in FIG. 5a or 5b. The preparation and setup of the photoresist coated wafer onto the immersion lithography system is performed as the first step 522 of the diagram. The light exposing operation to print the image pattern onto the photoresist of the production wafer utilizing the non surfactant-spiked water immersion lens fluid is performed as the next step 524. The immersion lithography printing is complete and the wafer is moved out of the immersion lens lithography system as step 526 of the diagram. The immersion lithography system may proceed with cleaning and rinsing of the water immersion lens and its fluid-material interfaces as the next step 528. After completion of the immersion lens cleaning procedures, the immersion lithography system may proceed to begin processing the next wafer as the diagram points back to restart with step 522. The immersion lens lithography system of this example diagram, FIG. 5b, utilizes surfactant-spiked immersion lens fluid as the lens cleaning fluid and the non surfactant-spiked immersion lens fluid to perform the image pattern printing. The previously disclosed valved, secondary immersion fluid supply and recovery systems help to facilitate the operations of this diagramed example.

As another alternative, a relatively diluted version of the surfactant-spiked immersion lens fluid can be used for the printing process since the less amount of “foreign” fluid contained in the water the better. After the printing process, a relatively stronger version of the surfactant-spiked immersion lens fluid can be injected for cleaning purposes so that a better cleaning process is guaranteed.

In short, when performing an immersion lithography process, a wafer is placed in the immersion lithography system, a light exposing operation is performed on the wafer using an objective lens immersed in a first fluid. Thereafter, the objective lens is cleaned using a surfactant containing second fluid. The first fluid may be a de-ionized water, and the second fluid may comprise NH₄OH, and may further comprise peroxide and/or water or ozone.

The disclosed method of using surfactant-spiked immersion lens fluid provides an effective means for the cleaning of the lens used within immersion lens lithography systems. The reduction of surface and interfacial tensions within the water immersion lens and their fluid-material interfaces modifies the behavior of the materials such that surfaces become more hydrophilic and that particulate defects tend to remain suspended within the water fluid medium. As a result, the particulate defects are quickly and easily purged away, as the adhesion, formation and growth of particulate defects and gas micro-bubbles within the water immersion lens fluid and fluid-material interfaces are greatly reduced. The method helps to maintain the integrity of the photoresist surface and pattern such that there is no dissociation/breakup, nor permanent stains or marks.

The present disclosure provides several examples to illustrate the flexibility of how surfactant-spiked water immersion lens fluid may be implemented. The disclosed surfactant-spiked fluid may be implemented as replacement for the conventional light projecting water immersion lens, or as a cleaning fluid solution used in conjunction with the conventional non surfactant-spiked water immersion lens fluids.

The disclosed method and featured surfactant-spiked water immersion lens fluid may be easily implemented into existing device process designs and flows as well as into their fabrication facilities and operations. The method and immersion lens fluid of the present disclosure may also be implemented into present advanced technology immersion lithography systems utilizing 197 nm to 250 nm exposing light wavelengths, as well as future systems utilizing light wavelengths smaller than 197 nm. The benefits provided by the disclosed methods and specified immersion lens fluid will allow for advanced technology semiconductor devices of high reliability, high performance and high quality.

The above disclosure provides many different embodiments or examples for implementing different features of the disclosure. Specific examples of components and processes are described to help clarify the disclosure. These are, of course, merely examples and are not intended to limit the disclosure from that described in the claims.

Although the invention is illustrated and described herein as embodied in a design and method for operating an immersion lithography system, it is nevertheless not intended to be limited to the details shown, since various modifications and structural changes may be made therein without departing from the spirit of the invention and within the scope and range of the equivalent of the claims. Accordingly, it is appropriate that the appended claims be construed broadly and in a manner consistent with the scope of the disclosure, as set forth in the following claims.

1. A method for cleaning lens used in an immersion lithography system (ILS), the method comprising:
positioning a wafer in the ILS; and
performing a light exposing operation on the wafer using an objective lens immersed in a first fluid containing surfactant.

2. The method of claim 1 wherein the wafer is coated with photoresist.

3. The method of claim 1 wherein the first fluid forms an immersion lens.

4. The method of claim 1 wherein the surfactant reduces a surface tension of the objective lens with the first fluid.

5. The method of claim 1 wherein the surfactant changes a surface property of the wafer to make it more hydrophilic.

6. The method of claim 1 further comprising cleaning the objective lens after the light exposing operation using a second fluid having a higher surfactant concentration than the first fluid.

7. The method of claim 6 further comprising providing the first fluid before starting the light exposing operation.

8. The method of claim 1 wherein the first fluid reduces floating defects including photoresist defects or micro-bubbles.

9. A method for cleaning lens used in an immersion lithography system (ILS), the method comprising:
positioning a wafer in the ILS;
performing a light exposing operation on the wafer using an objective lens immersed in a first fluid that does not contain surfactant; and
cleaning the objective lens using a second fluid comprising a surfactant-spiked water immersion fluid.

10. The method of claim 9 wherein the wafer is coated with photoresist.

11. The method of claim 9 wherein the first fluid is a de-ionized water.

12. The method of claim 9 wherein the surfactant is ionic.

13. The method of claim 9 wherein the surfactant is non-ionic.

14. The method of claim 9 wherein first and second fluids reduce floating defects including photoresist defects or micro-bubbles.

15. An immersion lithography system comprising:
means for positioning a wafer;
means for providing the first fluid containing no surfactant;
means for performing a light exposing operation on the wafer using an objective lens immersed in the first fluid; and
means for providing a surfactant to the first fluid to form a second fluid to reduce an adherence of floating defects to the wafer or the objective lens.

16. The system of claim 15 further comprising means for collecting the first fluid.

17. The system of claim 15 wherein the first fluid forms an immersion lens.

18. The system of claim 15 wherein the first fluid is de-ionized water.

19. The system of claim 15 further comprising means for collecting the second fluid.

20. A method for cleaning lens used in an immersion lithography system (ILS), the method comprising:
positioning a wafer in the ILS;
performing a light exposing operation on the wafer using an objective lens immersed in a first fluid; and
cleaning the objective lens using a second fluid containing surfactant.

21. The method of claim 20 wherein the wafer is coated with photoresist.

22. The method of claim 20 wherein the first fluid is a de-ionized water.

23. The method of claim 20 wherein the second fluid comprises NH₄OH.

24. The method of claim 23 wherein the second fluid further comprises peroxide (H₂O₂).

25. The method of claim 24 wherein the second fluid further comprises water.

26. The method of claim 20 wherein the second fluid comprises ozone (O₂)

27. The method of claim 20 wherein the second fluid comprises peroxide (H₂O₂).
